(19) United States
(12) Patent Application Publication
(10) Pub. No.: US 2021/0346520 A1
(43) Pub. Date: Nov. 11, 2021

(54) METHODS OF USING INTRAVENOUS REXIN-G: A TUMOR-TARGETED RETROVECTOR ENCODING A DOMINANT-NEGATIVE CYCLIN G1 (CCNG1) INHIBITOR FOR ADVANCED PANCREATIC CANCER

(71) Applicants: Erlinda M. Gordon, Santa Monica, CA (US); Frederick L. Hall, Carmel Valley, CA (US)

(72) Inventors: Erlinda M. Gordon, Santa Monica, CA (US); Frederick L. Hall, Carmel Valley, CA (US)

(21) Appl. No.: 17/348,736

(22) Filed: Jun. 15, 2021

Related U.S. Application Data
(63) Continuation of application No. PCT/US2019/066968, filed on Dec. 17, 2019.
(60) Provisional application No. 62/780,772, filed on Dec. 17, 2018.

(51) Int. Cl. A61K 48/00; A61K 38/18; A61K 45/06; A61K 31/704; A61K 31/4995; A61K 39/395; A61P 35/04

(52) U.S. Cl. CPC A61K 48/0058; A61K 38/18; A61K 45/06; A61K 31/704; A61P 35/04; A61K 39/3955; A61K 48/0083; A61K 48/0075; A61K 31/4995

(57) ABSTRACT

The present disclosure teaches methods of treating a patient who has an advanced metastatic cancer, after the patient has the patient has failed at least one treatment regimen for the advanced metastatic cancer, by administering a plurality of infusions of a vector comprising a tumor signature-targeting peptide and a nucleic acid that encodes a dominant negative human cyclin G1 construct. One or more of the patient's treatment regimens may have included gemcitabine. The present disclosure also provides methods of treatment by further administering to the patient an additional therapeutic agent such as an immune-modulatory monoclonal antibody, a cytotoxic chemotherapeutic agent, an anti-angiogenesis agent, a selective tyrosine kinase inhibitor, or a monoclonal antibody directed against specific features of cells from the metastatic cancer.

### RELATED CASES

This is a continuation of International Application PCT/US2019/066968 filed on Dec. 17, 2019, which claims priority to U.S. Provisional Patent Application No. 62/780,772, filed on Dec. 17, 2018, each of which is hereby incorporated by reference in its entirety.

### TECHNICAL FIELD

This disclosure relates generally to methods of treating cancer. More specifically, this disclosure relates to treatment of tumors in a patient with advanced pancreatic adenocarcinoma by administering a tumor-targeted vector encoding a dominant-negative cyclin G1 inhibitor.

### BACKGROUND

Pancreatic adenocarcinoma (PDAC) is projected to become the second leading cause of cancer death in the US and is rising worldwide^{1,2}. For patients with advanced disease, first^{3,4} and second line⁵ treatment options may improve survival modestly but are not curative. Unfortunately, there have been few successful targeted therapy options⁶ in part because the most common mutations (KRAS, TP53) have not been targetable, and others are uncommon (BRCA1, BRCA2, and MSI). As with most cancers, genetic dysfunction of the normal cell division cycle and its checkpoint control elements may be critical to progression of PDAC⁷; therefore, targeting the executive elements of cell cycle checkpoint control may represent a promising strategy⁸.

DeltaRex-G (Former names: Mx-dnG1, Rexin-G) is the first targeted injectable vector to be approved for clinical trials in the treatment of metastatic cancers⁹. DeltaRex-G (FIG. 1) is a non-replicative MLV-based amphotropic retrovector displaying a cryptic collagen-binding motif on its gp70 surface membrane that may target abnormal Signature (SIG™) proteins in the tumor microenvironment (TME)¹⁰, and encodes a dominant negative mutant construct (dnG1) of human cyclin GI (CCNG1)¹¹. The vector also contains a neomycin resistance (neo^{r}) gene which is driven by the SV40 early promoter. When injected intravenously, DeltaRex-G “seeks” out and accumulates in cancerous lesions by binding to exposed abnormal collagenous SIG proteins deposited as a result of tumor invasion. tumor-associated angiogenesis and stroma formation, elevating the vector concentration in the TME in the vicinity of cancer cells. Upon gaining entry into the rapidly proliferating cells within the TME¹². DeltaRex-G produces a cytocidal dnG1 protein that may block a pivotal check point of the cell division cycle, resulting in apoptosis, thus, elimination of cancer cells, proliferative tumor vasculature, and associated malignant fibroblasts¹³⁻¹⁴.

Based on encouraging clinical data from the Philippines in patients with metastatic PDAC¹⁵⁻¹⁶, clinical trials began in the United States using DeltaRex-G for standard chemotherapy-resistant PDAC, sarcoma, osteosarcoma, and breast cancer¹⁷⁻¹⁹. In this disclosure, we provide results, along with new mechanistic and pharmacological insights, from an advanced Phase I/II study evaluating over-all safety and potential antitumor activity of intravenous infusions of DeltaRex-G in metastatic gemcitabine-resistant PDAC.

Other viral gene therapy approaches for pancreatic adenocarcinoma include an on-going Phase 1 trial combining oncolytic adenovirus-mediated cytotoxic and IL-12 gene therapy with chemotherapy in metastatic PDAC (www.clinicaltrials.gov), and a recently completed Phase III randomized controlled clinical trial of PANVAC-VF for the treatment of patients with advanced pancreatic cancer. PANVAC™-VF is a vaccine regimen composed of a priming dose of recombinant vaccinia virus and booster doses of recombinant fowl pox virus expressing carcinoembryonic antigen, mucin-1 and a triad of costimulatory molecules (TRICOM), given subcutaneously, followed by injection of recombinant granulocyte-macrophage colony-stimulating factor at the vaccination site³¹⁻³². However, the Phase III randomized trial did not meet it primary endpoint of improving overall survival when compared with physician's choice of palliative therapy³³. Both viral gene therapies for PDAC involve either intratumor or subcutaneous viral vector injections. In contrast, DeltaRex-G involves a systemically (intravenously) administered tumor-targeted gene delivery approach (FIG. 1)^{10, 13-14, 18-20, 34, 43}

### BRIEF DESCRIPTION OF THE FIGURES

FIG. 1A is a graphic illustration showing a DeltaRex-G vector displaying a SIG targeting peptide for binding to Signature (Sig) Proteins in the tumor microenvironment (TME) and encoding a dominant negative human cyclin G1 inhibitor gene. Injected intravenously, DeltaRex-G nanoparticles seek out and bind to abnormal SIG proteins in the TME which augments effective vector concentration in tumors.

FIG. 1B is a graphic illustration showing tumor signature proteins, SIG-proteins, and tumor blood supply in a tumor microenvironment.

FIG. 1C is a graphic illustration of a DeltaRex-G vector encoding a dominant negative human cyclin G1 inhibitor gene.

FIG. 2 shows a Kaplan-Meier plot of progression-free survival in DeltaRex-G (Rexin-G)-treated gemcitabine-resistant PDAC. Progression-free survival data for the modified Intention-to-Treat population is displayed. The proportion of patients surviving progression-free is plotted on the vertical axis as a function of time from beginning of treatment, plotted on the horizontal axis.

FIG. 3A is a graphical representation of tumor regression during treatment with DeltaRex-G in Patient 012. Percentage change in tumor size (sum of longest diameter (SLD), is plotted on the vertical axis, as a function of time from beginning of DeltaRex-G (Rexin-G) treatment, plotted on the horizontal axis.

FIG. 3B is a graphical representation of tumor regression during treatment with DeltaRex-G in Patient 016. Percentage change in tumor size (sum of longest diameter (SLD), is plotted on the vertical axis, as a function of time from beginning of DeltaRex-G (Rexin-G) treatment, plotted on the horizontal axis.

FIG. 4A is a graphical representation of tumor regression during treatment with DeltaRex-G in Patient 018. Percentage change in tumor size (longest diameter, LD) of metastatic hepatic and lymph node sub-peritoneal lesions, are individually plotted on the vertical axis, as a function of time from beginning of DeltaRex-G (Rexin-G) treatment, plotted on the horizontal axis.

FIG. 4B is a graphical representation of CA-19.9 levels during treatment with DeltaRex-G in Patient 018. Serum levels of tumor marker CA 19.9 (U/mL) are plotted on the vertical axis, as a function of time from beginning of DeltaRex-G (Rexin-G) treatment, plotted on the horizontal axis.

FIG. 5 is a Kaplan Meier plot of overall survival of PDAC patients following DeltaRex-G treatment at escalating dose levels. Overall survival data for the Intention-to-Treat population is displayed. The proportion of patients surviving is plotted on the vertical axis as a function of time from beginning of treatment, plotted on the horizontal axis.

### DETAILED DESCRIPTION

DeltaRex-G is a potent inhibitor of the human Cyclin G1 Pathway (CCNG1 proto-oncogene). CCNG1 gene expression plays a powerful executive role in cell cycle regulation, exerting significant influence on critical oncogenic drivers: including the potent Mdm2 and cMyc oncoproteins, and the p53 tumor suppressor protein, gatekeeper of DNA fidelity³⁴. CCNG1 is overexpressed in over 50% of various malignancies: including pancreatic, breast, prostate, ovarian, and colon cancer³⁵. Albeit a small study in patient number, the single-agent antitumor activity of DeltaRex-G in metastatic pancreatic adenocarcinoma is evident. In addition to the single agent efficacy observed in the oncology clinic, molecular mechanisms were histologically revealed: as repeated intravenous infusion on DeltaRex-G induced apoptosis of cancer cells, stromal fibroblasts and associated tumor vasculature in biopsied tumors of DeltaRex-G treated patients^{19,36,37}. Conceivably, patients whose tumors overexpress CCNG1, revealing a pathological distortion in growth control pathways, will respond favorably to Cyclin G1 inhibitor therapy, delivered precisely. Without being bound by any particular theory, the DeltaRex-G induced tumor eradication by enforced apoptosis of cancer cells that was observed histologically, as well as supportive neo-vasculature and associated/malignant fibroblasts of the TME, is the executive mechanism of DeltaRex-G anticancer activity. Moreover, the demonstrated eradication of refractory chemo-resistant pancreatic cancer (that is, progressive eradication upon continued intravenous infusions) is certainly noteworthy and potentially important—prompting us to closely examine the anaplastic Signature (Sig) proteins with an aim toward “further optimizing” these pioneering aspects of tumor-targeted gene delivery in future investigations⁴⁴.

The present disclosure provides methods for treating a patient having advanced metastatic cancer, wherein the patient has failed at least one treatment regimen for the advanced metastatic cancer. In some embodiments, the patient has failed at least two treatment regimens. In certain embodiments, at least one treatment regimen comprised administration of gemcitabine. As a result, one or more tumors are resistant to gemcitabine or are otherwise resistant to certain first and/or second line therapeutic options. The methods comprise administering a plurality of intravenous infusions of a vector comprising a tumor signature-targeting peptide and a nucleic acid that encodes a dominant negative human cyclin G1 construct The vector may be DeltaRex-G.

The words “treating” and “treatment” have their usual meanings in medical science, that is, “treating” means the management and care of a patient to cure or alleviate a disease or disorder or one or more of the symptoms thereof. A treatment may achieve a “cure,” that is, a complete and permanent remission of a cancer, but it need not be a cure. Treatment may be undertaken to alleviate symptoms, for example, to decrease tumor size, the number and location of metastases, or the physiological effects of tumor burden. Treatment may lead to temporary remission or render the tumor more amenable to other therapeutic options (such as surgery, radiation, or treatment with a different therapeutic agent or combination of agents). It should also be noted that use of the terms “treating” and “treatment” is not meant to exclude other actions that may be necessary or desirable for the management and care of a cancer patient but that are not recited in the methods described in this disclosure, e.g., use of IV fluids for the patient's hydration or use of medications to treat pain.

In some embodiments, the vector may be administered in a 6-week cycle encompassing 4 weeks of treatment followed by 2 weeks of rest. The vector may be administered once weekly during the treatment weeks, twice weekly, or more frequently. Depending on the cancer's response and patient tolerance, the vector may be administered from between one cycle and thirteen cycles. In certain embodiments, the vector may be administered from between five cycles and thirteen cycles

In some embodiments, the vector dose may be between about 1×10¹¹ to about 500×10¹¹ colony forming units (cfu). In certain embodiments, the therapeutically effective dose is about 1×10¹¹ cfu, 5×10¹¹ cfu, 9×10¹¹ cfu, 22×10¹¹ cfu, 24×10¹¹ cfu, 30×10¹¹ cfu, 49×10¹¹ cfu, 60×10¹¹ cfu, 156×10¹¹ cfu, 314×10¹¹ cfu, or 453×10¹¹ cfu.

In some embodiments, the patient may have metastatic pancreatic adenocarcinoma. The patient may have at least one lesion in the pancreas, liver, lymph nodes, lung, trachea, adrenal glands, mesentery, bone, or omentum. In other embodiments, the patient may further display malignant ascites, pleural effusion, and/or peritoneal carcinomatosis.

In some embodiments, the patient may be administered one or more additional therapeutic agents, for example, an immune-modulatory monoclonal antibody, a cytotoxic chemotherapy, an anti-angiogenesis agent, a selective tyrosine kinase inhibitor, or a monoclonal antibody directed against specific features of cells from the metastatic cancer. In certain embodiments, the additional therapeutic agent comprises an immune-modulatory monoclonal antibody. In a subset of such embodiments, the therapeutic agent may be one or more checkpoint inhibitors. In certain other embodiments, the additional therapeutic agent comprises a cytotoxic chemotherapy agent. In a subset of such embodiments, the therapeutic agent may be doxorubicin, trabectedin, other known chemotherapy agent, or combination thereof. In certain other embodiments, the additional therapeutic agent comprises an anti-angiogenesis agent. In a subset of such embodiments, the therapeutic agent may be bevacizumab. In certain other embodiments, the additional therapeutic agent comprises a selective tyrosine kinase inhibitor. In certain other embodiments, the additional therapeutic agent comprises one or more monoclonal antibodies directed against specific features of cells from the metastatic cancer. In a subset of such embodiments, the therapeutic agent may be panitumumab, cetuximab, or a combination thereof.

It will be readily understood that the embodiments, as generally described herein, are exemplary. This detailed description of various embodiments is not intended to limit the scope of the present disclosure but is merely representative of various embodiments.

### EXAMPLES

The following examples are for illustration only. In light of this disclosure, those of skill in the art will recognize that variations of these examples and other embodiments of the disclosed subject matter are enabled without undue experimentation.

### Example 1

### Vector and FDA-Approved Vector Production

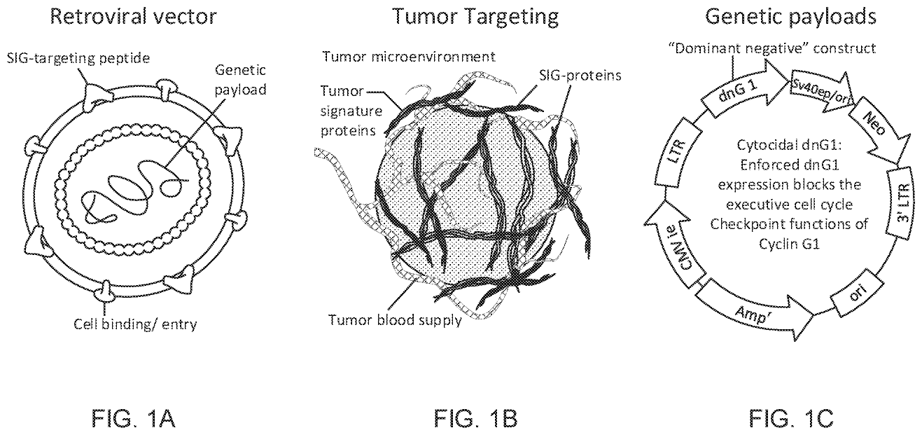

DeltaRex-G (FIG. 1) is a non-replicative MLV-based amphotropic retrovector displaying a cryptic collagen-binding motif on its gp70 surface membrane that can target abnormal Signature (Sig) proteins in the tumor microenvironment (TME)^{10,44} and encodes a dominant negative mutant construct (dnG1) of human cyclin G1 (CONG1)¹¹. The vector also contains a neomycin resistance (neo^{r}) gene which can be driven by the SV40 early promoter. The DeltaRex-G vector can be produced by transient co-transfection of human embryonic kidney 293 T cells. Clinical vector production and characterization have been described elsewhere¹⁵⁻¹⁷. The final product exhibits a vector titer of 5×10⁹ colony forming units (cfu) per milliliter, a biologic potency of 50-70% growth inhibitory activity in target cancer cells, less than 550 bp residual DNA, no detectable E1A or SV40 large T antigen, and no detectable replication competent retrovirus (RCR), in compliance with FDA recommendations for retroviral vector-based gene therapy products. The vector formulation was stored in aliquots of 23 ml in a 30 ml glass vial and kept frozen at −70° to −90° C. until used. Preparation of the DeltaRex-G vector for patient administration consisted of rapid thawing of the vector in the vial or a cryobag in a 34° C. water bath. The vector was thawed 15 to 30 minutes prior to infusion into the patient and given intravenously at 4 ml/per minute^{18,19}. All personnel who handled and disposed of the vector observed Biosafety Level 2 compliance in accordance with the National Institutes of Health Guidelines for Research Involving Recombinant DNA molecules.

### Example 2

### Vector-Related Testing and Biodistribution Analysis

Detection of anti-vector antibodies in serum, testing for presence of replication competent retrovirus and vector DNA integration studies in patient peripheral blood lymphocytes, were performed as described previously¹⁷.

### Example 3

### Study Design

This was an open label, single aim, dose-seeking study that incorporated a modification of the standard Cohort of 3 design, that allowed patients to continue the study drug into Phase II¹⁸⁻¹⁹. Treatment with DeltaRex-G comprised 6-week cycles that encompassed 4 weeks of treatment, followed by 2 weeks of rest. Four dose levels were given, beginning at 1.0×10¹¹ cfu given by intravenous (i.v.) infusion two times per week. Three patients were to be treated at each dose level with expansion to 6 patients per cohort if dose-limiting toxicity (DLT) was observed in any 1 of the first 3 patients at each dose level. The maximum tolerated dose (MTD) was defined as the highest dose in which 0 of 3 or ≤1 of 6 patients experienced a DLT, with the next higher dose level having at least 2 patients who experienced a DLT. A DLT was defined as any National Cancer Institute Common Toxicity Criteria for Adverse Events (CTCAE) Grade 3, 4, or 5 adverse event (AE) considered possibly, probably, or definitely related to the study drug, excluding the following: Grade 3 absolute neutrophil count lasting<72 hours: Grade 3 alopecia; or any Grade 3 or higher incident of nausea, vomiting, or diarrhea in a patient who did not receive maximal supportive care³⁹.

For the Phase II part of the study, patients who had no toxicity or in whom toxicity had resolved to Grade 1 or less could receive additional cycles of therapy. Protocol Amendments I and II permitted an intra-patient dose escalation up to Dose Level II for patients who had no toxicity or in whom toxicity had resolved to Grade 1 or less, once safety had been established at the higher dose level in a simultaneously conducted Phase I/II study for sarcoma¹⁸. Additionally, each cohort also could be expanded to 6 or 7 patients if significant biologic activity (stable disease or better) was noted at each dose level. The principal investigator was allowed to recommend surgical resection/debulking after at least one treatment cycle has been completed. Response was evaluated first using the Response Evaluation Criteria in Solid Tumors⁴⁰. Additional evaluations used the International Positron Emission Tomography (PET) criteria⁴¹ and a modified RECIST as described by Choi et al.⁴². Safety and efficacy analyses were conducted by the site Principal Investigators.

### Example 4

### Patient Population and Treatment

Inclusion Criteria: Candidates included in the study had to have a histologically or cytologically confirmed pathologic diagnosis of advanced or metastatic pancreatic adenocarcinoma that was resistant to gemcitabine or a gemcitabine—containing regimen, be ≥18 years of age, have an Eastern Cooperative Oncology Group (ECOG) performance score of 0-1, and acceptable hematologic, hepatic, and kidney function.

Exclusion criteria included human immunodeficiency vims, hepatitis B virus, or hepatitis C virus positivity, clinically significant ascites, medical or psychiatric conditions that could compromise proper adherence to the protocol, and unwillingness to employ effective contraception during treatment with DeltaRex-G and for 6 weeks following treatment completion.

The clinical protocol was reviewed and approved by the Western Institutional Review Board, Olympia, Wash. The patients were recruited on a first-come first-serve basis and a written informed consent was obtained from each patient at the time of enrollment. All personnel who handled and disposed of the vector observed Biosafety Level 2 compliance in accordance with the National Institutes of Health Guidelines for Research Involving Recombinant DNA molecules.

This Phase I/II trial enrolled 20 patients with metastatic gemcitabine-refractory pancreatic adenocarcinoma. Table 1 shows the patient demographics. The patients had failed a median of two regimens, one of which contained gemcitabine. All patients exhibited metastatic disease. Two patients had one target lesion, and 17 patients had 2-7 target lesions in the pancreas, lymph node, omentum, mesentery, adrenal, bone, lung, and the liver in 16 patients (Table 2). Aside from the target lesions in Table 2, all patients had either many non-target lesions, malignant ascites, pleural effusion, and peritoneal carcinomatosis. Therefore, target lesions alone may not reflect the patients' total tumor burden.

TABLE 1
Patient demographics.

| Age | |
|---|---|
| Median | 62 |
| Range | 50-83 |
| Gender | |
| Female | 12 (60%) |
| Male | 8 (40%) |
| Race | |
| White | 16 (80%) |
| Asian | 4 (20%) |
| Disease Stage | |
| Metastatic (1-7 target lesions + non-target | 20 (100%) |
| lesions, ascites, pleural effusion, peritoneal | |
| carcinomatosis) | |
| Performance Score (ECOG) | 20 (100%) |
| 1 | |
| Number of Previous Chemotherapy Regimens | |
| Median | 2 |
| Range | 1-7 |

TABLE 2
Locations and sizes of lesions in 20 patients with metastatic PDAC.

| | LOCATION OF TARGET LESIONS |
|---|---|
| SUBJECT ID | (Size, longest diameter, mm) |
| 001 | Left periumbilical (85) |
| 002 | Preaortic node (40) |
| | R lobe liver, post aspect (27) |
| | Pancreas (20) |
| | Celiac node (11) |
| 003 | R lobe liver, upper anterior aspect (23) |
| | Body of Pancreas (22) |
| | Anterior R lower lobe, liver (17) |
| | Anterior R lobe liver, porta hepatis (18) |
| | Posterior to porta hepatis (22) |
| | Inferior tip of R liver lobe (11) |
| 004 | L Liver lobe, lateral tip (32) |
| | L Liver lobe (36) |
| | Mid R Liver lobe (17) |
| | Body of Pancreas (10) |
| 005 | L lobe liver at splenic hilum (76) |
| | R lobe liver, lateral dome (22) |
| | Liver, segment 4A (58) |
| | Anterior omentum (42) |
| | L lung, hilar (27) |
| | R pre-tracheal node (10) |
| 006 | Perihilar R ML, lung (10) |
| | L lobe liver, lateral segment (35) |
| | Posterior R lobe, liver (41) |
| | L lobe liver, lateral segment (29) |
| | Lateral L lobe liver (26) |
| | Pancreatic tail (22) |
| | L adrenal apex (11) |
| 007 | R hepatic lobe, posterior (88) |
| | Right hepatic lobe, upper portion (45) |
| 008 | R Liver Lobe Post. Aspect (24) |
| | L Liver Lobe (55) |
| | Anterior to L Kidney (11) |
| | Slightly Lower to SI (19) |
| | Level of SI 1st Lesion (12) |
| | Level of SI 2nd Lesion (14) |
| 009 | Lateral tip L liver lobe (14) |
| | Inferior anterior margin, L liver lobe (16) |
| | R anterior subcapsular (23) |
| | R liver lobe dome (19) |
| | L liver lobe anterior superficial subcapsular (10) |
| 010 | RLL Lung Posterior Subpleural Upper (12) |
| | RLL Lung Posterior Subpleural Lower (15) |
| 011 | Anterior segment 5, liver (64) |
| | Segment 4 liver (48) |
| | Pancreatic body, preaortic (52) |
| 012 | R Liver lobe, posterior (21) |
| | Head of Pancreas medial to biliary stent (32) |
| 013 | Tail of Pancreas (66) |
| | Left adrenal (10) |
| | Mid segment 4, liver (40) |
| 014 | Dome R hepatic lobe (20) |
| | Lateral tip L hepatic lobe (18) |
| | Head of Pancreas (30) |
| | Nodule anterior abdominal wall to R of midline |
| | at T12 (12) |
| 015 | Head & body of Pancreas (67) |
| | Mesenteric mass slightly L of midline (19) |
| | Circular lesion posterior to porta hepatis (34) |
| | R hepatic lobe anterior & superior to gall bladder (36) |
| 016 | Head of Pancreas (90) |
| | Aortocaval mass at R hepatic lobe (20) |
| 017 | Pancreatic Head (28) |
| 018 | Subhepatic area below Anastomosis (28) |
| | Subperitoneal lymph node (22) |
| 019 | R Posterior sulcus lung (15) |
| B01 | Lat. L Lower Lobe Lung (14) |
| | Medial L lower lobe lung (21) |
| | R of Hepatic Art. (13) |
| | L periaortic lymph node (15) |
| | L adrenal nodule (11) |

Dose Escalations: Six patients were treated at Dose Levels 0-I; 7 were treated at Dose Level II; and 7 were treated at Dose Level III. One patient was included in the Dose II cohort because he received an (FDA-approved) intrapatient dose escalation from Dose 0 to Dose II. The number of DeltaRex-G infusions, the number of completed cycles of DeltaRex-G, and the total exposure (cfu) to DeltaRex-G are summarized by dosage group in Table 3.

The median number of infusions varied from 9 in Dose Group 0-I to 52 in Dose Group III. A total of 832 infusions were administered for all patients. The total number of completed infusion cycles varied from 5 in Dose Group 0-I to 31 in Dose Group III. The median cumulative dose of DeltaRex-G increased from 9×10¹¹ cfu in Dose Group 0-I to 60×10¹¹ cfu in Dose Group II to 156×10¹¹ cfu in Dose Group III. Total exposure to DeltaRex-G for all patients was 1927×10¹¹ cfu, with a range from 30 to 453×10¹¹ cfu.

TABLE 3
Total exposure to DeltaRex-G in 20 patients with metastatic PDAC.
Dose Group for Analysis

| | 0-I | II | III | AII |
|---|---|---|---|---|
| Parameter | (N = 6) | (N = 7) | (N = 7) | (N = 20) |
| Number of all infusions | | | | |
| Total for all | 72 | 356 | 404 | 832 |
| patients | | | | |
| Median (min, max) | 9 (5, 23) | 30 (11, 157) | 52 (10, 151) | 24 (5, 157) |
| per patient | | | | |
| Number of all | | | | |
| completed cycles | | | | |
| Total for all | 5 | 27 | 31 | 63 |
| patients | | | | |
| Median (min, max) | 2 (1, 2) | 3 (1, 13) | 4 (1, 12) | 3 (1, 13) |
| per patient | | | | |
| CFU of DeltaRex-G | | | | |
| Total (×10e11 cfu) | 73 | 676 | 1212 | 1961 |
| Median (min, mx) | 9 (5, 24) | 60 (22, 314) | 156 (30, 453) | 49 (5, 453) |
| (×10e11 cfu) per patient | | | | |

### Example 5

### Evaluation of Tumor Burden

Estimated tumor burden was determined for each patient using the following formula:

ETB (# cancer cells)=[Sum of Target Lesions (cm)+(No. of Non-Target Lesions+(20*)]×10⁹ (Assumption: 1 cm=1×10⁹ cancer cells).

*Note: 20×10⁹ cancer cells for each occurrence of ascites, pleural effusion, and/or ‘too many to count’ non-target lesion.

### Example 6

### Safety Analysis

Pretreatment evaluation included history, physical exam, complete blood count with differential and platelet count, a serum chemistry panel including aspartate transaminase, alanine transaminase, alkaline phosphatase, creatinine, and total bilirubin, assessment of coagulation status including prothrombin time, international normalized ratio, and activated partial thromboplastin time, testing for human immunodeficiency virus, hepatitis B virus, and hepatitis C virus. All patients had a complete blood count and serum chemistry panel performed weekly during treatment. Toxicity was evaluated before each vector infusion, as well as before beginning an additional treatment cycle. Toxicity was graded using NCI CT-CAE version 3³⁹. Patients' serum was collected for detection of vector neutralizing antibodies and antibodies to gp70. The peripheral blood mononuclear cells were also collected to test for the presence of vector DNA integration and RCR at the end of 4 weeks, at 6 weeks, or before the start of a treatment cycle. Vector-related studies were performed as previously described¹⁸. DeltaRex-G was stored in volumes of 23 ml in 30 ml vials or 40 ml in 150 ml cryobags at −80° C. Preparation of the vector for patient administration consisted of rapid thawing in the vial in a 34° C. water bath 15-30 minutes prior to infusion and was given intravenously over 5-10 minutes. All personnel who handled and disposed of the vector observed Biosafety Level 2 compliance in accordance with the National Institutes of Health Guidelines for Research Involving Recombinant DNA molecules.

There were no dose-limiting toxicities observed at any dose level. Unrelated adverse events were reported for all 20 patients. Related but clinically non-significant adverse events occurred in 7 patients and all were Grade 1 (Table 4). These comprised of chills (1 patient), fatigue (2 patients) and headache (1 patient) at Dose Level II, and fatigue (4 patients) at Dose Level III. There was no treatment-related loss of hair, nausea, vomiting, anemia, thrombocytopenia, neutropenia, liver, lung or kidney dysfunction reported. There were no serious drug-related AEs.

TABLE 4
Clinically non-significant drug-related adverse events by dose level
and toxicity grade (n = 20).

| | Preferred | Dose | Toxicity |
|---|---|---|---|
| MedRA System Organ Class | Term | Level | Grade |
| General disorders and administration | Fatigue | 2 | 1 |
| site conditions | | | |
| General disorders and administration | Fatigue | 2 | 1 |
| site conditions | | | |
| Musculoskeletal and connective | Chills | 2 | 1 |
| tissue disorders | | | |
| Nervous system disorders | Headache | 2 | 1 |
| General disorders and administration | Fatigue | 3 | 1 |
| site conditions | | | |
| General disorders and administration | Fatigue | 3 | 1 |
| site conditions | | | |
| General disorders and administration | Fatigue | 3 | 1 |
| site conditions | | | |
| General disorders and administration | Fatigue | 3 | 1 |
| site conditions | | | |

The most frequent clinically non-significant unrelated Grade 3 AEs were hypoalbuminemia (4 patients) and increased alanine aminotransferase (3 patients). Anemia, hyperglycemia, increased aspartate aminotransferase and hypocalcemia were reported in 2 patients each. Other clinically non-significant unrelated Grade 3 AEs were reported in 1 patient each. Several types of unrelated adverse events appeared to be more frequent at higher doses: anemia, hyperbilirubinemia, increased aspartate aminotransferase and decreased appetite (Table 5). Thirteen patients experienced 25 serious adverse events, all of which were deemed not related to the study drug. Details regarding these AEs are provided in Table 6.

TABLE 5
Clinically non-significant, unrelated, grade 3 adverse events reported in
≥2 patients by DeltaRex-G Dose Level.

| | Dose Level | | | | |
|---|---|---|---|---|---|
| | N = 6 | | N = 7 | N = 7 | |
| MedRA System Organ | 0 | I | II | III | Total |
| Class/Preferred Term | N = 2 | N = 4 | N = 7 | N = 7 | N = 20 |
| Blood and lymphatic system disorders | | | | | |
| Anemia | 1 | | | 1 | 2 |
| Endocrine disorders | | | | | |
| Hyperglycemia | | | 1 | 1 | 2 |
| Hepatobiliary disorders | | | | | |
| Hypoalbuminemia | | 1 | | 3 | 4 |
| Investigations | | | | | |
| Alanine aminotransferase increased | 1 | | 1 | 1 | 3 |
| Aspartate aminotransferase | 1 | 1 | | | 2 |
| increased | | | | | |
| Blood alkaline phosphatase | | | | 2 | 2 |
| increased | | | | | |
| Metabolism and nutrition disorders | | | | | |
| Hypocalcemia | 1 | | 1 | | 2 |

TABLE 6
Serious unrelated adverse event listings by dose level and
toxicity grade

| | | MedDRA | Dose | Toxicity |
|---|---|---|---|---|
| Patient | System Organ Class | Preferred Term | Level | Grade |
| 1 | Gastrointestinal disorders | Obstruction gastric | 0 | 3 |
| | Gastrointestinal disorders | Intestinal perforation | 0 | 3 |
| 2 | Neoplasms benign, | Malignant pleural | 0 | 3 |
| | malignant and unspecified | effusion | | |
| 3 | Infection and infestations | Sepsis | 1 | 3 |
| 4 | Injury, poisoning and | Overdose | 1 | 3 |
| | procedural complications | | | |
| 5 | Gastrointestinal disorders | Abdominal pain | 1 | 3 |
| 6 | Vascular disorders | Pulmonary | 2 | 3 |
| | | embolism | | |
| 7 | Metabolism and nutrition | Dehydration | 2 | 3 |
| | disorders | | | |
| | Blood and lymphatic | Thrombocytopenia | 2 | 4 |
| | system disorders | | | |
| | Metabolism and nutrition | Hyponatremia | 2 | 4 |
| | disorders | | | |
| 8 | Gastrointestinal disorders | Obstruction gastric | 2 | 3 |
| 9 | Vascular disorders | Pulmonary | 3 | 3 |
| | | embolism | | |
| | Infection and infestations | Sepsis | 3 | 3 |
| 10 | Gastrointestinal disorders | Fecaloma | 3 | 3 |
| | Gastrointestinal disorders | Constipation | 3 | 3 |
| | Metabolism and nutrition | Hyponatremia | 3 | 4 |
| | disorders | | | |
| | Endocrine disorders | Hyperglycemia | 3 | 4 |
| 11 | Hepatobiliary disorders | Cholangitis | 3 | 3 |
| 12 | Psychiatric disorders | Mental status | 3 | 3 |
| | | changes | | |
| | Gastrointestinal disorders | Ascites | 3 | 3 |
| | Nervous system disorders | Altered state of | 3 | 3 |
| | | consciousness | | |
| 13 | Respiratory, thoracic and | Pneumothorax | 3 | 3 |
| | mediastinal disorders | | | |
| | Psychiatric disorders | Delirium | 3 | 3 |
| | Metabolism and nutrition | Dehydration | 3 | 3 |
| | disorders | | | |
| | Investigations | Aspartate | 3 | 4 |
| | | aminotransferase | | |
| | | increased | | |

No patient tested positive for any of the following: vector neutralizing antibodies, antibodies to gp70, replication-competent retrovirus in peripheral blood lymphocytes (PBLs); vector integration into genomic DNA of PBLs.

To date, 19 out of the 20 patients enrolled in the study have died. None of the deaths were considered related to DeltaRex-G. The cause of death was progressive disease in all but one patient for whom the cause of death was sepsis. Remarkably, the long-term survivor exhibited lymphatic metastasis prior to intravenous DeltaRex-G infusions as salvage therapy.

### Example 7

### Efficacy Analysis

Prior to beginning treatment, imaging evaluations such as whole body FDG/PET-CT scan, electrocardiography, and chest X-ray were performed. FDG PET-CT scan was done for efficacy assessment at the end of 4 weeks, at the end of 6 weeks, or before starting an additional treatment cycle up to 12 weeks, and every 12 weeks thereafter. RECISTv1.0 criteria was used to assess the tumor responses [complete response (CR); Partial response (PR); or Stable Disease (SD)]⁴⁰. Tumor control rate was defined as the percentage of patients who had CR, PR or SD at any time during the DeltaRex-G treatment period. Tumor responses were also evaluated using modifications of the International PET criteria⁴¹ and the CHOI criteria⁴². The modified International PET Criteria defines a CR as disappearance of FDG avid uptake in target and non-target lesions with no new lesions; PR as a decrease in maximum standard uptake value of >25% from baseline with no new lesions along with no obvious progression of non-target lesions; PD as an increase in maximum standard uptake value of >25% from baseline, any new lesions, and obvious progression of non-target lesions; and SD as not meeting the criteria for CR, PR, or PD, and no symptomatic deterioration attributed to tumor progression. The modified CHOI criteria defines CR as the disappearance of all disease and no new lesions; PR as a decrease in size of ≥10% or a decrease in CT density (Hounsfeld units)≥15% with no new lesions and no obvious progression of non-measurable disease; PD as an increase in tumor size of >10% and did not meet criteria for PR by CT density, any new lesions, including new tumor nodules in a previously cystic tumor; and SD as not meeting the criteria for CR, PR, or PD, and no symptomatic deterioration attributed to tumor progression.

Of the 20 enrolled patients, fifteen received at least one complete cycle (4 weeks) of treatment and had a follow-up PET-CT scan and therefore, were considered evaluable for efficacy (modified Intent-to-Treat or mITT population) in terms of response, progression-free survival and overall survival. In the first cohort (Dose Level I), three patients were withdrawn from the study prior to completion of one treatment cycle either due to disease-related complications (n=1; worsening malignant pleural effusion) or due to a personal decision to discontinue treatment (n=2; one patient had worsening ascites, and the other decided to take alternative medicine). In the second cohort (Dose Level II), one patient had worsening ascites and clinical deterioration, and in the third cohort (Dose Level III), one patient had worsening malignant pleural effusion.

Table 7 shows evaluation of tumor response using RECIST v1.0, Choi and modified international PET Criteria in the mITT population. In the overall cohort, the median tumor burden was 32.6×10⁹ cells; the range in tumor burden was wide across patients, with a minimum of 5.0×10⁹ cells and a maximum of 115.5×10⁹ cells. Notably, patients at Dose Level III had significantly larger tumor loads (52.1×10⁹ cancer cells) than those in Dose Group 0-I or II (32.6×10⁹ and 31.5×10⁹ cancer cells respectively). Patients were assigned to dose levels on a first come first served basis. No significant relationship was noted between estimated tumor burden and response/PFS/OS.

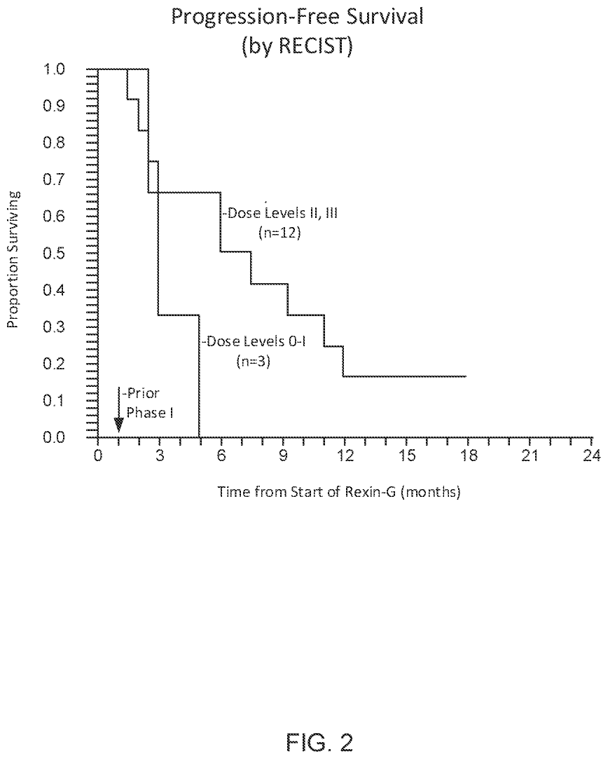

By RECIST, one patient achieved a CR, two patients had a PR and 12 had SD. The tumor control rate (CR+PR+SD) by RECISTv1.0 was 100% (15/15 patients). Responses were more frequent when assessed using modified international PET criteria or Choi Criteria. By PET, one patient achieved a CR, 4 patients had a PR, and 10 patients had SD. By Choi, one patient had a CR, 5 had a PR and 8 had SD. One patient did not have a Choi analysis because the lesions were too small. By RECIST, PRs and CRs occurred only at Dose Levels II and III, suggesting a dose-dependent relationship between DeltaRex-G dose and response. PFS by RECIST was 2.7, 4.0, and 5.6 months at Dose Levels 0-I, II, and III respectively, suggesting a dose-dependent relationship between DeltaRex-G dose and PFS. Kaplan-Meier analysis of progression free survival (FIG. 2) in the mITT group suggests a trend toward a dose-response relationship between progression-free survival and DeltaRex-G dosage. It is important to note that a higher tumor burden was observed for patients in Doses II and III compared with Doses 0-I providing evidence in support of DeltaRex-G's antitumor activity.

TABLE 7
Summary of responses, tumor burden, progression-free survival and
overall survival.

| | Dose Level^{a,} | | | |
|---|---|---|---|---|
| Category | 0-I | II | III | AII |
| miTT Patients ^{b} | N = 3 | N = 6 | N = 6 | N = 15 |
| Median Tumor Burden | 13.5 | 37.1 | 38.8 | ND |
| (# × 10e9 cancer cells) | | | | |
| Best Response | | | | |
| RECIST v1.0 (n = 15) | 3SD | 1 PR; 5SD | 1CR; 1PR; | 1 CR; 2PR; |
| | | | 4SD | 12SD |
| PET (n = 15) | 1 PR; | 1 PR; 5SD | 1CR; 2PR; | 1CR; 4PR; |
| | 2SD | | 3SD | IOSD |
| Choi (n = 14) | 1 PR; | 2PR; 4SD | 1CR; 3PR; | 1CR; 5PR; |
| | 2SD | | 1SD | 8SD |
| Median PFS (mo.) | | | | |
| RECIST | 2.7 | 4.0 | 5.6 | ND |
| PET | 2.7 | 4.1 | 4.2 | ND |
| Choi | 2.7 | 4.1 | 6.9 | ND |
| Median OS (mo.) | 4.3 | 9.2 | 9.2 | ND |
| % OS | | | | |
| 1 year | 0 | 33.3% | | |
| 1.5 years | | 25.0% | | |
| ITT population^{c} | N = 6 | N = 7 | N = 7 | N = 20 |

Abbreviations: ITT, intent-to-treat; mITT, modified ITT; RECIST v 1.0, Response Evaluation Criteria in Solid Tumors; PET, positron emission tomography; Choi, modified RECIST as described by Choi et al.¹; PFS, progression-free survival; OS, overall survival; cum, cumulative; mo., month; ND, not determined
^{a}Dose Level 0 = 1 × 10¹¹ cfu BIW; Dose Level I = 1 × 10¹¹ cfu TIW; Dose Level II = 2 × 10¹¹ cfu TIW; Dose Level III = 3 × 10¹¹ cfu TIW
^{b} mITT population defined as all patients who received at least one cycle and had a follow-up PET CT scan.
^{c}ITT population defined as all patients who received at least one infusion of DeltaRex-G;
^{d} As of Dec. 1, 2018

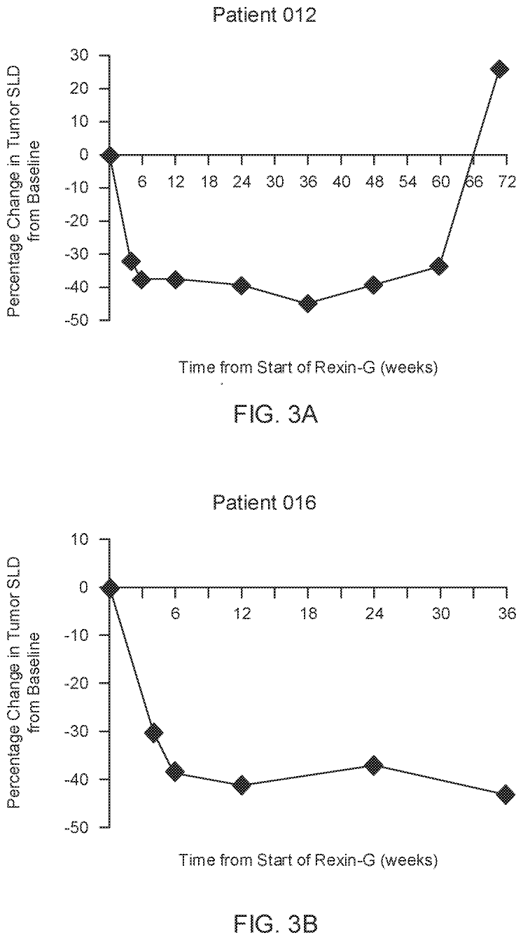

Table 8 shows tumor response in three patients with durable tumor response patterns when assessed by RECIST, Choi and PET (Patient 012, Patient 016, and Patient 018). Patient 012-CJP, is a 56-year-old white female, s/ρ biliary stent placement and radiation therapy for poorly differentiated PDAC, who failed gemcitabine, and had target lesions at the pancreatic head medial to the biliary stent, and right liver lobe. She achieved a best response of PR by RECIST at Week 4 and continued in PR through Week 36 (FIG. 3A). She had definitive disease progression at Week 60. Patient 016-JLM, a 59-year-old white female with poorly differentiated PDAC had failed four chemotherapy regimens, including gemcitabine, 5-FU, oxaliplatin and capecitabine with target lesions at the head of pancreas and an aortocaval mass by right hepaticlobe. She achieved a best response of PR by RECIST at Weeks 4, 6, 12, 24, and 36 (FIG. 3B). She discontinued treatment with DeltaRex-G on Week 42 due to an AE (bile duct obstruction). She withdrew from the study due to symptomatic progression without a confirmatory CT scan. Patient 018 is a white female (72 years of age at study entry) who had been initially diagnosed with non-metastatic, poorly differentiated adenocarcinoma of the pancreas, underwent a Whipple's resection with postoperative radiation therapy, and received chemotherapy with 5-FU and gemcitabine for one year. A year later, she presented with hepatic and lymph node metastases (target lesions), and mesenteric stranding indicative of peritoneal carcinomatosis (non-target lesions) with a rising serum CA19.9 level. She was advised to receive further chemotherapy, but decided in favor of participating in the Phase I/II study using DeltaRex-G. The patient completed a total of 17.9 months of therapy with DeltaRex-G and did not achieve CR until Week 36 of treatment; this patient has remained in CR at the time of study completion. Notably, when examined separately, the two target lesions were found to have different disappearance profiles. As shown in FIG. 3A, the lymph node metastasis decreased more rapidly, starting at Week 6, than the hepatic metastasis, which increased in size before ultimately completely resolving in Week 36. Serum levels of the tumor marker CA-19.9 decreased by 45%, from 76 to 42 U/mL (normal level is <37 U/mL) by Week 19, and then remained relatively constant thereafter (FIG. 3B). She received no additional chemotherapy or alternative treatment after discontinuation of DeltaRex-G therapy and remains in sustained remission with no evidence of disease or late onset adverse events as of November 2018.

TABLE 8
Notable tumor response patterns at each assessment point for
patients with partial and complete responses by RECIST
vI.0 compared to Choi and PET criteria.

| | | Overall | Overall | Overall |
|---|---|---|---|---|
| | Assessment | Response | Response | Response |
| Patient | Time Point | by RECIST | by Choi | by PET |
| 012 | Week 4 | PR | SD | SD |
| | Week 6 | PR | SD | PR |
| | Week 12 | PR | PR | PR |
| | Week 24 | PR | SD | SD |
| | Week 36 | PR | SD | PD |
| | Week 48 | SD | PD | PD |
| | Week 60 | PD | PD | PD |
| | Week 72 | PD | PD | PD |
| 016 | Week 4 | PR | SD | SD |
| | Week 6 | PR | SD | SD |
| | Week 12 | PR | PR | SD |
| | Week 24 | PR | PR | PD |
| | Week 36 | PR | PR | PD |
| 018 | Week 4 | SD | SD | PD |
| | Week 6 | SD | PR | PD |
| | Week 12 | SD | SD | SD |
| | Week 24 | PR | PR | PR |
| | Week 36 | CR | CR | CR |
| | Week 48 | CR | CR | CR |
| | Week 60 | CR | CR | CR |
| | Week 72 | CR | CR | CR |

Of note, continuation of treatment contributed to efficacy/clinical benefit in at least five patients. These patients survived from 10.6 months to 10 years after starting DeltaRex-G. Anti-tumor effects differed for individual target lesions in some patients. These data suggest that patients may benefit from extended treatment: with DeltaRex-G despite signs of apparent progression (pseudoprogression), which may result from the known mechanism of action of DeltaRex-G: induction of apoptosis via cell cycle blockade of cancer cells, tumor vasculature, and malignant tumor associated fibroblasts without bone marrow suppression, which may initially cause lesions to appear larger due to inflammatory or immunologic responses seen in published reports^{39, 42-43.}

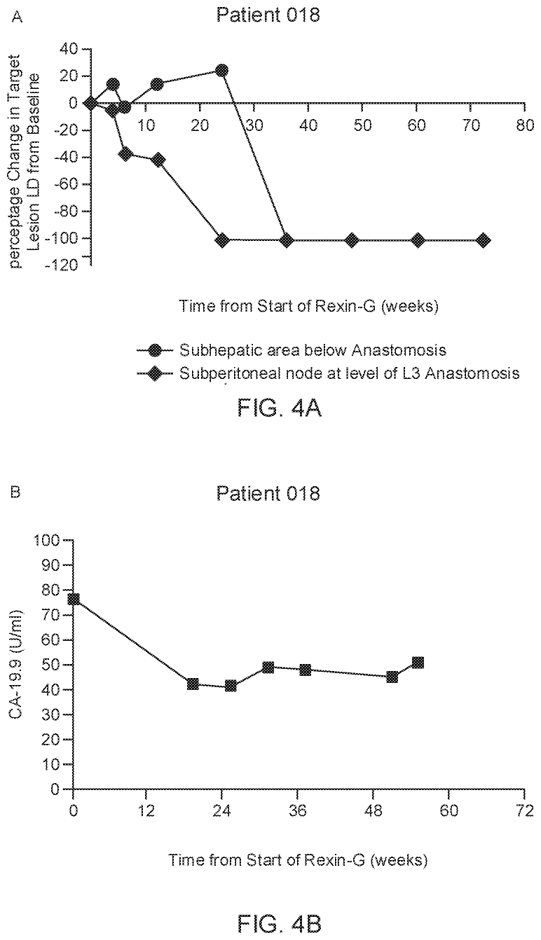

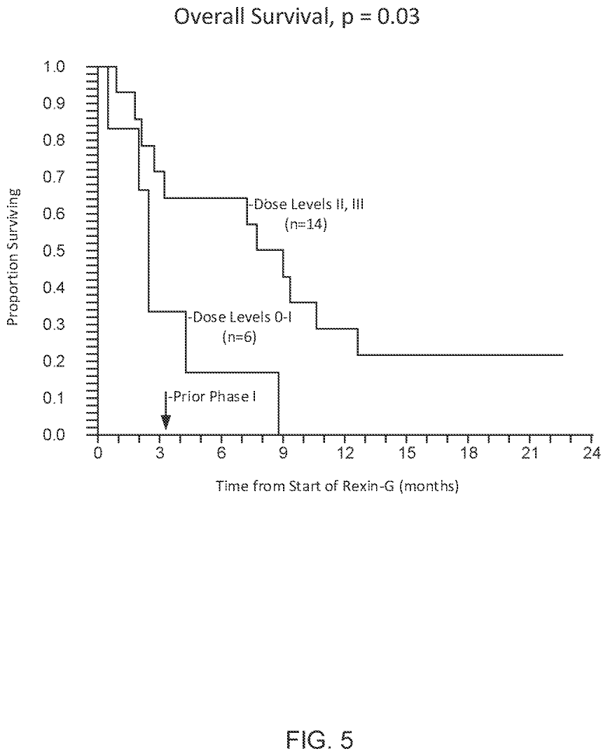

Median overall survival in the mITT group was calculated to be 4.3 months at Dose 0-I, 9.2 months at Dose II and 9.2 months at Dose III. The OS estimates in the efficacy evaluable mITT population among the combined group of Dose Levels 0-I was 0% at one year. In contrast, OS estimates in the combined groups Dose Levels II-III were 33.3% at one year and 25% at 1.5 years. The median OS in the ITT group was 2.6 months in the Dose 0-I cohort vs 9.0 and 7.8 months in the Dose II and III cohorts respectively. The OS rates in the Dose 0-I group was 0% at one year. In contrast, OS rates among the combined group of Dose II-III were 28.6% 1 year and 21.4% at 1.5 years (p=0.03) compared with Dose 0-I. Kaplan-Meier analysis of overall survival in the ITT population suggests a dose-response relationship between overall survival and DeltaRex-G dosage (p=0.03; FIG. 4).

Using the one-sided Fisher Test, we compared tumor control responses (TCR by RECIST) in this advanced Phase I/II study (n=15; TCR 1 CR, 2 PR, 12 SD) with those in the prior Phase I study where patients received up to a total dose of 6×10¹¹ CFU per cycle (TCR 1 SD, 11 PD)¹⁴. With “tumor control response” designated as CR, PR, or SD at any given time during DeltaRex-G treatment period, the proportions are 15/15 for the current study and 1/12 in the prior study, with p<0.0001 by the one-sided Fisher test. These data indicate a dose-response relationship between tumor control response (TCR) and DeltaRex-G dosage across studies.

Discussion: This report updates and extends a Phase I/II study of safety and efficacy using DeltaRex-G in gemcitabine-refractory PDAC with additional analysis and new mechanistic insights. The initial clinical data was previously reported on 13 patients by Chawla et al.¹⁹ Safety was established with no DLT following multiple DeltaRex-G infusions at all four dose levels, and the MTD was not reached. It is important to note that there was no treatment-related loss of hair, bone marrow suppression nor organ dysfunction at all dose levels. The serious adverse events experienced by these patients were probably due to disease-related complications, and not to DeltaRex-G treatment as assessed by the principal investigators. Further, there were no vector-related safety issues raised, as evidenced by no detected anti-vector neutralizing antibodies, antibodies to gp70, replication-competent retrovirus in PBLs nor vector integration into genomic DNA of PBLs. These data suggest the safety of DeltaRex-G when compared to FDA-approved therapies forepeak such as nab-paclitaxel, gemcitabine, FOLFORINOX, and erlotinib^{3,4,6,21-30}. Regarding efficacy, we report durable response rates (3/15; 20%) lasting 36-72 weeks during DeltaRex-G treatment. Two patients were progression-free for more than one year: Patient 012 had a PFS of 13.8 months; Patient 018 had a PFS>17.9 months. The best overall response rates (20%) noted in this study were significantly better than those reported by Galanis et al.¹⁷ which used much lower DeltaRex-G doses. In support of this observation, a significant dose response relationship was shown between overall survival and DeltaRex-G dose in the Intent-to-Treat population. Remarkably, one patient is still alive 10 years later with no evidence of PDAC. The documented eradication of cancer within the lymphatic system has compelling implications that warrant additional studies of the anaplastic Signature (Sig) proteins involved⁴⁴.

In conclusion, the clinical data gleaned from this Phase I/II study of precision, tumor-targeted genetic medicine suggests that (a) DeltaRex-G is exceptionally safe with a wide margin of safety, and (b) DeltaRex-G exhibits dose-dependent antitumor activity in patients with gemcitabine-refractory metastatic PDAC. Based on the analysis of clinical data, DeltaRex-G gained fast track designation from the USFDA for the conduct of a planned Phase II/III study using the optimal Dose Level III treatment schedule of DeltaRex-G versus physician's choice in a larger number of patients. This planned Phase II/III study may include correlations of CCNG1 gene expression in tumors, along with pertinent companion diagnostics, histology, and treatment outcome parameters.

### REFERENCES

All references cited in this disclosure are incorporated by reference in their entirety.
1. Rahib L, Smith B D, Aizenberg R, Rosenzweig A B, Fleshman J M and Matrisian L M: Projecting cancer incidence and deaths to 2030: The unexpected burden of thyroid, liver, and pancreas cancers in the United States. Cancer Res. 74: 2913-2921, 4142014. https://doi.org/10.1158/0008-5472.CAN-14-0155 PJ\lflD:24840647
2. American Cancer Society (2018). Cancer facts & figures 2018. https://www.cancer.org/content/dam/cancer-org/research/cancer-facts-and-statistics/annual-cancer-facts-and-figures/2018/cancer-facts-and-figures-2018.pdf
3. Conroy T., Desseigne F., Ychou M., Bouché O., Guimbaud R., Bécouarn Y., Adenis A., Raoul J. L., Gourgou-Bourgade S., de la Fouchardière C., Groupe Tumeurs Digestives of Unicancer. PRODIGE Intergroup FOLFIRINOX versus gerncitabine for metastatic pancreatic cancer. N Engl J Med 364: 1817-1825, 2011. https://doi.org/10.1056/NEJMoal0 11923PM ID:21561347
4. Von Hoff D D, Ervin T, Arena F P, Chiorean E G, Infante J, Moore M, Seay T, Tjulandin S A, Ma W W, Saleh M N, et al: Increased survival in pancreatic cancer with nab-paclitaxel plus gemcitabine. N Engl J Med 369: 1691-1703, 2013. https://doi.org/10.1056/NEJMoal304369P MID:2413I 140
5. Wang-Gillam A, Li C P, Bodoky G, Dean A, Shan Y S, Jameson G, Macarulla T, Lee K H, Cunningham D, Blanc J F, et al; NAPOLI-1 Study Group: Nanoliposomal irinotecan with fluorouracil and folinic acid in metastatic pancreatic cancer after previous gemcitabine-based therapy (NAPOLI-1): A global, randomised, open-label, phase 3 trial. Lancet 387: 545-557, 2016. https://doi.org/10.1016/S0140-6736(15)00986-I PMID:26615328
6. Moore M J, Goldstein D, Hamm J, Figer A, Hecht J R, Gallinger S, Au H J, Murawa P, Walde D, Wolff R A, et al; National Cancer Institute of Canada Clinical Trials Group: Erlotinib plus gemcitabine compared with gemcitabine alone in patients with advanced pancreatic cancer: A phase III trial of the National Cancer Institute of Canada Clinical Trials Group. J Clin Oncol 25: 1960-1966, 2007. https://doi.org/10. I 200/JCO.2006.07.9525 PMID:17452677
7. Liu Y, Elsawa S F, Almada L L. Primers on molecular pathways—cycling toward pancreatic cancer. Pancreatology. 2010; 10(1):6-13.
8. Matera R, Saif M W. New therapeutic directions for advanced pancreatic cancer: cell cycle inhibitors, stromal modifiers and conjugated therapies. Expert Opin Emerg Drugs. 2017 September; 22(3):223-233.
9. Waehler R, Russell S J and Curiel D T: Engineering targeted viral vectors for gene therapy. Nat Rev Genet 8: 573-587, 2007. https://doi.org/10.1038/mg2141 PMID:17607305
10. Hall F L, Liu L, Zhu N L, Stapfer M, Anderson W F, Beart R W, et al. Molecular engineering of matrix-targeted retroviral vectors incorporating a surveillance function inherent in von willebrand factor. Hum Gene Ther. 2000; 11 (7):983-93.
11. Xu F, Prescott M F, Liu P X, Chen Z H, Liau G, Gordon E M, et al. Long term inhibition of neointima formation in balloon-injured rat arteries by intraluminal instillation of a matrix-targeted retroviral vector bearing a cytocidal mutant cyclin g1 construct. Int J Mol Med. 2001; 8 (1):19-30
12. Gordon E M and Hall F L. Rexin-G: A Targeted Genetic Medicine for Cancer. Expert Opin Biol Ther 10:819-832, 2010
13. Gordon E M, Liu P X, Chen Z H, Liu L, Whitley M D, Gee C, Groshen S, Hinton D R, Beart R W and Hall F L: Inhibition of metastatic tumor growth in nude mice by portal vein infusions of matrix-targeted retroviral vectors bearing a cytocidal cyclin G1 construct. Cancer Res 60: 3343-3347, 2000. PMID:10910035
14. Gordon E M, Chen Z H, Liu L, Whitley M, Liu L, Wei D, Groshen S, Hinton D R, Anderson W F, Beart R W Jr, et al: Systemic administration of a matrix-targeted retroviral vector is efficacious for cancer gene therapy in mice. Hum Gene Ther 12: 193-204, 2001. https://doi.org/10.1089/104303401750061258 PMID:11177556
15. Gordon E M, Cornelio G H, Lorenzo C C III, Levy J P, Reed R A, Liu Land Hall F L: First clinical experience using a ‘pathotropic’ injectable retroviral vector (Rexin-G) as intervention for stage IV pancreatic cancer. Int J Oncol 24: 177-185, 2004. PMID:14654955
16. Gordon E M, Lopez F F, Cornelio G H, Lorenzo C C III, Levy J P, Reed R A, Liu L, Bruckner H W and Hall F L: Pathotropic nanoparticles for cancer gene therapy Rexin-G IV: Three-year clinical experience. Int J Oncol 29: 1053-1064, 2006. PMID:17016635
17. Galanis E, Carlson S K, Foster N R, Lowe V, Quevedo F, McWilliams R R, Grothey A, Jatoi A, Alberts S R and Rubin J: Phase I trial of a pathotropic retroviral vector expressing a cytocidal cyclin G1 construct (Rexin-G) in patients with advanced pancreatic cancer. Mol Ther 16: 979-984, 2008. https://doi.org/10.1038/mt.2008.29 PMID:18388964
18. Chawla S P, Chua V S, Fernandez L, Quon D, Saralou A, Blackwelder W C, Hall F L and Gordon E M: Phase I/II and phase II studies of targeted gene delivery in vivo: Intravenous Rexin-G for chemotherapy-resistant sarcoma and osteosarcoma. Mol Ther 17: 1651-1657, 2009. https://doi.org/10.1038/mt.2009.126 PMID:19532136
19. Chawla S P, Chua V S, Fernandez L, Saralou A, Quon D, Blackwelder W B, Gordon E M. Hall F L. Advanced Phase I/II studies of targeted gene delivery in vivo: Intravenous Rexin-G for gemcitabine-resistant metastatic pancreatic cancer. Mol Ther 18: 435-441: 2010
20. Kim S, Federman N, Gordon E M, Frederick H L, Chawla S P, Rexin-G®, a tumor-targeted retrovector for malignant peripheral nerve sheath tumor: A case report.
21. Burris H A III, Moore M J, Andersen J, Green M R, Rothenberg M L, Modiano M R, Cripps M C, Portenoy R K, Storniolo A M, Tarassoff P, et al: Improvements in survival and clinical benefit with gemcitabine as first-line therapy for patients with advanced pancreas cancer: A randomized trial. J Clin Oncol 15: 2403-2413, 1997. https://doi.org/10.1200/JCO.1997.15.6.2403 PMID:9196156
22. Berlin J D, Catalano P, Thomas J P, Kugler J W, Haller D G and Benson A B III: Phase III study of gemcitabine in combination with fluorouracil versus gemcitabine alone in patients with advanced pancreatic carcinoma: Eastern Cooperative Oncology Group Trial E2297. J Clin Oncol 20: 3270-3275, 2002. https://doi.org/10.1200/JCO.2002.11.149 PMID:12149301
23. Rocha Lima C M, Green M R, Rotche R, Miller W H Jr, Jeffrey G M, Cisar L A, Morganti A, Orlando N, Gruia G and Miller L L: Irinotecan plus gemcitabine results in no survival advantage compared with gemcitabine monotherapy in patients with locally advanced or metastatic pancreatic cancer despite increased tumor response rate. J Clin Oncol 22: 3776-3783, 2004. https://doi.org/10.1200/JCO.2004.12.082 PMID:15365074
24. Heinemann V, Quietzsch D, Gieseler F, Gonnermann M, Schönekäs H, Rost A, Neuhaus H, Haag C, Clemens M, Heinrich B, et al: Randomized phase III trial of gemcitabine plus cisplatin compared with gemcitabine alone in advanced pancreatic cancer. J Clin Oncol 24: 3946-3952, 2006. https://doi.org/10.1200/JCO.2005.05.1490 PMID:16921047
25. Colucci G, Giuliani F, Gebbia V, Biglietto M, Rabitti P, Uomo G, Cigolari S, Testa A, Maiello E and Lopez M: Gemcitabine alone or with cisplatin for the treatment of patients with locally advanced and/or metastatic pancreatic carcinoma: A prospective, randomized phase III study of the Gruppo Oncologia dell'Italia Meridionale. Cancer 94: 902-910, 2002. https://doi.org/10.1002/cncr.10323 PMID:11920457
26. Louvet C, Labianca R, Hammel P, Lledo G, Zampino M G, André T, Zaniboni A, Ducreux M, Aitini E, Taïeb J, et al: GERCOR; GISCAD: Gemcitabine in combination with oxaliplatin compared with gemcitabine alone in locally advanced or metastatic pancreatic cancer: Results of a GERCOR and GISCAD phase III trial. J Clin Oncol 23: 3509-3516, 2005. https://doi.org/10.1200/JCO.2005.06.023 PMID:15908661
27. Poplin E, Feng Y, Berlin J, Rothenberg M L, Hochster H, Mitchell E, Alberts S, O'Dwyer P, Haller D, Catalano P, et al: Phase III, randomized study of gemcitabine and oxaliplatin versus gemcitabine (fixed-dose rate infusion) compared with gemcitabine (30-minute infusion) in patients with pancreatic carcinoma E6201: A trial of the Eastern Cooperative Oncology Group. J Clin Oncol 27: 3778-3785, 2009. https://doi.org/10.1200/JCO.2008.20.9007 PMID:19581537
28. Herrmann R, Bodoky G, Ruhstaller T, Glimelius B, Bajetta E, Schüller J, Saletti P, Bauer J, Figer A, Pestalozzi B, et al; Swiss Group for Clinical Cancer Research; Central European Cooperative Oncology Group: Gemcitabine plus capecitabine compared with gemcitabine alone in advanced pancreatic cancer: A randomized, multicenter, phase III trial of the Swiss Group for Clinical Cancer Research and the Central European Cooperative Oncology Group. J Clin Oncol 25: 2212-2217, 2007. https://doi.org/10.1200/JCO.2006.09.0886 PMID:17538165
29. Burris H III and Rocha-Lima C: New therapeutic directions for advanced pancreatic cancer: Targeting the epidermal growth factor and vascular endothelial growth factor pathways. Oncologist 13: 289-298, 2008. https://doi.org/10.1634/theoncologist.2007-0134 PMID:18378539
30. Senderowicz A M, Johnson J R, Sridhara R, Zimmerman P, Justice R and Pazdur R; Erlotinib/gemcitabine for first-line treatment of locally advanced or metastatic adenocarcinoma of the pancreas. Oncology (Williston Park) 21: 1696-1709, 1712, 1715, 2007. PMID: 18247017
31. Schuetz T, Kaufman H L, Marshall J L, Safran H; Extended survival in second-line pancreatic cancer after therapeutic vaccination, June 2005, Journal of Clinical Oncology 23(16), DOI: 10.1200/jco.2005.23.16_suppl.2576
32. Petrulio C A, Kaufman H L; Development of the PANVAC™-VF vaccine for pancreatic cancer, Expert Review of Vaccines, (2006) 5:1, 9-19, DOI: 10.1586/14760584.5.I. 9
33. Therion Reports Results of Phase 3 panvac-vf Trial and Announces Plans for Company Sale. PR Newswire 28 Jun., 2018. Available online: www.pmewswire.com
34. Gordon E M, Ravicz J R, Liu S, Chawla S P and Hall F L: Cell cycle checkpoint control: The cyclin G1/Mdm2/p53 axis emerges as a strategic target for broad-spectrum cancer gene therapy—A review of molecular mechanisms for oncologists. Mol Clin Oncol 9:115-134, 2018.
35. Ravicz J, Liu S, Andrali S S, Reddy S K, Sellappan S, Leong B, Chawla S P, Hall F L, and Gordon E M; Differential expression of human cyclin G1 (CCNG1) in cancer: A novel biomarker in development for CCNGI inhibitor therapy. J Clin Oncol 36, 2018 (suppl; abstr e24315).
36. Hall F L, Levy J P, Reed R A, Petchpud W N, Chua V S, Chawla S P and Gordon E M. Pathotropic targeting advances clinical oncology: Tumor-targeted localization of therapeutic gene delivery. Oncol Rep 24:829-833, 2010.
37. Gordon E M and Hall F L: Rexin-G, a targeted genetic medicine for cancer. Expert Opin Biol Ther 10: 819-832, 2010. https://doi.org/10.1517/14712598.2010.481666PMID:20384524
38. FDA guidance for industry: Supplemental guidance on testing for replication competent retrovirus in retroviral vector based gene therapy products and during follow-up of patients in clinical trials using retroviral vectors 2006: Available from: http://www.fda.gov/downloads/BiologicsBloodVaccines/GuidanceComplianceRegula toryInformation/Guidances/CellularandGeneTherapy/ucm078723.pdf
39. The NCI Common Terminology Criteria for Adverse Events Version 3 (2006). Cancer Therapy Evaluation Program DCTD, NCI, NIH, DHHS; 1-72, 2003. <http://ctep.cancer.gov>.
40. Therasse P, Arbuck S G, Eisenhauer E A, Wanders J, Kaplan R S, Rubinstein L, Ve1weij J, Van Glabbeke M, van Oosterom A T, Christian M C, et al: New guidelines to evaluate the response to treatment in solid tumors. European Organization for Research and Treatment of Cancer, National Cancer Institute of the United States, National Cancer Institute of Canada. J Natl Cancer Inst 92: 205-216, 2000. https://doi.org/10.1093/jnci/92.3.205 PMID:10655437
41. Young H, Baum R, Cremerius U, Herholz K, Hoekstra 0, Lammertsma A A, Pruim J and Price P; European Organization for Research and Treatment of Cancer (EORTC) PET Study Group: Measurement of clinical and subclinical tumour response using [18F]-fluorodeoxyglucose and positron emission tomography: Review and 1999 EORTC recommendations. Eur J Cancer 35: 1773-1782, 1999. https://doi.org/10.1016/S0959-8049(99)00229-4 PMID:10673991
42. Choi H, Chamsangavej C, Faria S C, Macapinlac H A, Burgess M A, Patel S R, Chen L L, Podoloff D A and Benjamin R S: Correlation of computed tomography and positron emission tomography in patients with metastatic gastrointestinal stromal tumor treated at a single institution with imatinib mesylate: Proposal of new computed tomograph response criteria. J Clin Oncol 25: 1753-1759, 2007. https://doi.org/10.1200/JCO.2006.07.3049 PMID:17470865
43. Stendahl Dy P, Chawla S P, Hall F L and Gordon E M. Immune Cell Trafficking in the Tumor microenvironment of human cyclin G1 (CCNG1) inhibitor-treated tumors. Brit J Cancer Res 1:4, 2018. doi: 10.31488/bjcr.117.
44. Gordon E M, Hall F L. PCT patent: Targeting the signature (Sig) of invading tumors using SIG binding peptides, Original Filing Date: Feb. 4, 2017.

1. A method of treating a patient having advanced metastatic cancer, wherein the patient is suffering from one or more lesions that are resistant to gemcitabine or gemcitabine-containing regimens, the method comprising administering a plurality of infusions of a vector comprising a tumor signature-targeting peptide and a nucleic acid that encodes a dominant negative human cyclin G1 construct.

2. The method of claim 1, wherein the vector is DeltaRex-G.

3. The method of claim 1 or 2, wherein the vector is administered in a 6-week cycle comprising 4 weeks of treatment followed by 2 weeks of rest.

4. The method of claim 3, wherein the vector is administered from between 1 and about 13 cycles.

5. The method of claim 4, wherein the vector is administered from between about 5 and about 13 cycles.

6. The method of any of claims 1-5, wherein the vector is administered at a dose of between about 1×10¹¹ and about 5×10¹³ cfu per infusion.

7. The method of any of claims 1-6, wherein the vector is administered at a dose of about 1×10¹¹ cfu, about 5×10¹¹ cfu, about 9×10¹¹ cfu, about 22×10¹¹ cfu, about 24×10¹¹ cfu, about 30×10¹¹ cfu, about 49×10¹¹ cfu, about 60×10¹¹ cfu, about 156×10¹¹ cfu, about 314×10¹¹ cfu, or about 453×10¹¹ cfu per infusion

8. The method of any of claims 1-7, wherein the advanced metastatic cancer is metastatic pancreatic adenocarcinoma.

9. The method of claim 8, wherein the patient has at least one lesion in the pancreas, liver, lymph nodes, lung, trachea, adrenal glands, mesentery, bone, or omentum.

10. The method of claim 8 or 9, wherein the patient has at least one of malignant ascites, pleural effusion, or peritoneal carcinomatosis.

11. A method of treating a patient having advanced metastatic cancer, wherein the patient has failed at least one treatment regimen for the advanced metastatic cancer, the method comprising administering a plurality of infusions of a vector comprising a tumor signature targeting peptide and a nucleic acid that encodes a dominant negative human cyclin G1 construct.

12. The method of claim 11, wherein the patient has failed at least two treatment regimens.

13. The method of claim 11 or 12, wherein at least one treatment regimen comprised administration of gemcitabine to the patient.

14. The method of claim 12 or 13, wherein the vector is administered in a 6-week cycle comprising 4 weeks of treatment followed by 2 weeks of rest.

15. The method of claim 14, wherein the vector is administered from between 1 and about 13 cycles.

16. The method of claim 14, wherein the vector is administered from between about 5 and about 13 cycles.

17. The method of any of claims 11-16, wherein the vector is administered at a dose of between about 1×10¹¹ and about 5×10¹³ cfu per infusion.

18. The method of any of claims 11-17, wherein the vector is administered at a dose of about 1×10¹¹ cfu, about 5×10¹¹ cfu, about 9×10¹¹ cfu, about 22×10¹¹ cfu, about 24×10¹¹ cfu, about 30×10¹¹ cfu, about 49×10¹¹ cfu, about 60×10¹¹ cfu, about 156×10¹¹ cfu, about 314×10¹¹ cfu, or about 453×10¹¹ cfu per infusion

19. The method of any of claims 11-18, wherein the advanced metastatic cancer is metastatic pancreatic adenocarcinoma.

20. The method of claim 19, wherein the patient has at least one lesion in the pancreas, liver, lymph nodes, lung, trachea, adrenal glands, mesentery, bone, or omentum.

21. The method of claim 19 or 20, wherein the patient has at least one of malignant ascites, pleural effusion, or peritoneal carcinomatosis.

22. The method of any of claims 1-21, further comprising the step of administering to the patient a therapeutic agent that is selected from the group consisting of immune-modulatory monoclonal antibodies, cytotoxic chemotherapies, anti-angiogenesis agents, selective tyrosine kinase inhibitors, and monoclonal antibodies directed against specific features of cells from the metastatic cancer.

23. The method of claim 22, wherein the therapeutic agent comprises an immune-modulatory monoclonal antibody.

24. The method of claim 23, wherein the therapeutic agent comprises a checkpoint inhibitor.

25. The method of claim 22, wherein the therapeutic agent comprises a cytotoxic chemotherapy.

26. The method of claim 25, wherein the therapeutic agent is selected from the group consisting of doxorubicin and trabectedin.

27. The method of claim 22, wherein the therapeutic agent comprises an anti-angiogenesis agent.

28. The method of claim 27, wherein the therapeutic agent comprises bevacizumab.

29. The method of claim 22, wherein the therapeutic agent comprises a selective tyrosine kinase inhibitor.

30. The method of claim 22, wherein the therapeutic agent comprises a monoclonal antibody directed against specific features of cells from the metastatic cancer.

31. The method of claim 30, wherein the therapeutic agent is selected from the group consisting of panitumumab and cetuximab.

32. The method of any of claims 11-21, wherein the vector is DeltaRex-G.

33. The method of claim 3 or 14, wherein the vector is administered at least once weekly to the patient during the weeks of treatment.

34. The method of claim 33, wherein the vector is administered twice weekly to the patient during the weeks of treatment.
